(19) United States
(12) Patent Application Publication
(10) Pub. No.: US 2025/0367151 A1
(43) Pub. Date: Dec. 4, 2025

(54) ANTI-VWF THERAPEUTIC FOR PREVENTING ARTERIAL THROMBI

(71) Applicant: GEORGIA TECH RESEARCH CORPORATION, Atlanta, GA (US)

(72) Inventors: David N. KU, Atlanta, GA (US); Christopher BRESETTE, Atlanta, GA (US)

(21) Appl. No.: 18/875,512

(22) PCT Filed: Jun. 16, 2023
(86) PCT No.: PCT/US2023/025575

§ 371 Date: Dec. 16, 2024

Related U.S. Application Data
(60) Provisional application No. 63/366,472, filed on Jun. 16, 2022.

(51) Int. Cl. A61K 31/198; A61K 31/095; A61K 31/194; A61K 38/06; A61P 7/02

(52) U.S. Cl. CPC A61K 31/198; A61K 31/095; A61K 31/194; A61K 38/063; A61P 7/02

(57) ABSTRACT

Disclosed are methods and compositions for treating or preventing arterial thrombosis in a subject in need thereof comprising administering to the subject a thiol or selenium containing compound.

### CROSS REFERENCE TO RELATED APPLICATIONS

This application claims the benefit of priority to U.S. Provisional Application 63/366,472, filed Jun. 16, 2022, which is incorporated by reference herein in its entirety.

### BACKGROUND

Ischemic strokes and heart attacks are commonly caused by arterial thrombi blocking blood flow through an artery. These ischemic events are a leading cause of death in the U.S., responsible for nearly ⅕ of all deaths (Virani S S, et al. Heart disease and stroke statistics-2020 update: A report from the American Heart Association. Circulation. 2020, 139-596). The clots that form in arterial conditions are unlike the well-studied coagulation clots and are therefore resistant to treatment with anti-coagulants and fibrin directed lytic agents (Martinez de Lizarrondo S, et al. Potent Thrombolytic Effect of N-Acetylcysteine on Arterial Thrombi. Circulation. 2017;136:646-60). In contrast, these clots are platelet rich and formed through a mechanism known as shear-induced platelet accumulation (SIPA) (Casa L D C, et al., Thrombus Formation at High Shear Rates. Annu Rev Biomed Eng. 2017;19(1):415-33). During SIPA, platelets can bind vWF, a protein found in blood plasma, and release additional vWF, rapidly forming a network of platelet aggregates that can span a stenotic artery in less than an hour.

Previous therapeutics to reduce the risk of arterial thrombosis have targeted platelet activation. The goal of those drugs is to reduce platelet activation, limiting the release of additional vWF into the plasma and preventing the positive feedback system from starting (Armstrong E J, et al. Association of dual-antiplatelet therapy with reduced major adverse cardiovascular events in patients with symptomatic peripheral arterial disease. J Vasc Surg. 2015;62(1):157-65). However, platelet activation also plays a large role in amplifying the coagulation cascade and the benefits of anti-platelet drugs must be weighed against increased risk of serious bleeding they cause (Hansen M, et al., Risk of Bleeding With Single, Dual, or Triple Therapy With Warfarin, Aspirin, and Clopidogrel in Patients With Atrial Fibrillation. Orig Investig. 2010;170(16):1433-41; Costa F, et al. Derivation and validation of the predicting bleeding complications in patients undergoing stent implantation and subsequent dual antiplatelet therapy (PRECISE-DAPT) score: a pooled analysis of individual-patient datasets from clinical trials. Lancet. 2017;389(10073):1025-34). Moreover, the clinical efficacy of these antiplatelet agents to prevent thrombi is only about 5%, with an additional 5% of patients experiencing severe bleeding from these agents leading many cardiologists to question whether the efficacy/safety profile is suitable for wide-spread use (Id.)

Because arterial thrombosis accounts for the largest number of fatalities in the U.S., preventing these thrombotic occlusions remains a long-standing, economically important problem with few solutions. The compositions and methods disclosed herein provide address these needs.

### SUMMARY

In accordance with the purposes of the disclosed materials and methods, as embodied and broadly described herein, the disclosed subject matter, in one aspect, relates to compounds, compositions and methods of making and using compounds and compositions. In some aspects, the techniques described herein relate to a method of treating or preventing arterial thrombosis in a subject in need thereof, including: administering to the subject a thiol containing compound. In some aspects, the techniques described herein relate to a method, wherein the thiol containing compound is N-acetyl cysteine. In some aspects, the techniques described herein relate to a method, wherein the thiol containing compound is cysteine, dithiothreitol, selenocysteine, glutathione, dimercaptosuccinic acid, thioterpinol, methanethiol, ethanethiol, or any combination thereof. In some aspects, the techniques described herein relate to a method, wherein the thiol containing compound is a peptide including N-acetyl cysteine. In some aspects, the techniques described herein relate to a method, wherein the thiol containing compound is administered at from 1 to 10 mM. In some aspects, the techniques described herein relate to a method, wherein the thiol containing compound is administered at from 3 to 5 mM. In some aspects, the techniques described herein relate to a method, wherein the thiol containing compound is administered at from 100 to 1000 mg/kg dose. In some aspects, the techniques described herein relate to a method, wherein the thiol containing compound is administered at from 400 mg/kg dose. In some aspects, the techniques described herein relate to a method, wherein the thiol containing compound is administered once. In some aspects, the techniques described herein relate to a method, wherein the subject is at high risk of forming arterial thrombi by having a blood concentration of vWF in an upper 4th quartile. In some aspects, the techniques described herein relate to a method, wherein the subject is hospitalized for cardiovascular disease. In some aspects, the techniques described herein relate to a method, wherein the subject is undergoing angioplasty or percutaneous coronary intervention, has a heart attack, or has ischemic stroke. In some aspects, the techniques described herein relate to a method of reducing thrombi formation in an artery, including: administering to the artery an effective amount of a thiol containing compound. In some aspects, the techniques described herein relate to a method of treating or preventing arterial thrombosis in a subject in need thereof, including: administering to the subject a selenium containing compound. In some aspects, the techniques described herein relate to a method of reducing thrombi formation in an artery, including: administering to the artery an effective amount of a selenium containing compound. In some aspects, the techniques described herein relate to a method, wherein the selenium containing compound is selenocysteine.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects and together with the description serve to explain the principles of the invention.

FIGS. 1A-1C show the addition of NAC to human whole blood increases occlusion times in a microfluidic model of arterial thrombosis. FIG. 1A: Administration of 3 mM and 5 mM NAC results in higher occlusion times. At 10 mM no occlusion is measured (* p<0.01, ** p<0.001). FIG. 1B: 4 parameter logistic fit of occlusion times vs NAC dose, r²=0.782. FIG. 1C: Increasing NAC dose leads to improved survival times in the microfluidic experiment. (* p<0.001)

FIGS. 2A-2D show platelet intensities of growing thrombi. FIG. 2A: Representative images of control clot (left and 10 mM channel with no platelet accumulation (right). Flow is from bottom to top. FIG. 2B: Representative plot of platelet intensities vs. time for one individual. The black arrow indicates the control lag time. FIG. 2C: Measured lag times for each condition, (** p<0.0001). FIG. 2D: Platelet aggregation rates normalized to each subject's control, (* p<0.05).

FIG. 3 shows strong NAC response is correlated with higher OTs and can be predicted by WBC count. Top: Strong NAC responders were defined as individuals with OTs for 5 mM significantly higher than OTs for 3 mM. These individuals also had significantly higher OTs than weak responders for all conditions in which an occlusive clot was formed (* p<0.01, ** p<0.0001). Bottom: ROC curve for using WBC count to predict the strength of an individual's response to NAC (AUC=0.92). Individuals with WBC counts under 6.3×10³/μL were significantly more likely to be strong NAC responders.

FIG. 4 shows blood treated with 10 mM NAC forms occlusive thrombi after addition of 19 IU/mL VWF. Top: Occlusion times of 10 mM with and without addition of 19 IU vWF, demonstrating recovery of the ability to form occlusive thrombi. Bottom: Survival curves for the VWF recovery study (* p<0.001).

FIGS. 5A-5C show in vivo occlusion times and clot stability. FIG. 5A: Average blood flow measurements following crush injury. Mice treated with vehicle form stable occlusions while mice treated with NAC form occlusive thrombi that recanalize (** p<0.01). FIG. 5B: 400 mg/kg NAC completely prevented occlusion in a murine model of arterial thrombosis and 200 mg/kg NAC trended towards higher occlusion times compared to the vehicle injections (* p<0.05). FIG. 5C: Clots formed with 200 mg/kg NAC were more likely to be unstable and recanalize during the experiment compared to the control.

FIGS. 6A-6C show the effect of repeat administration of NAC. FIG. 6A: Occlusion time for repeat injection conditions. 0-0 is PBS at t=−6 and t=0, 200-200 is 200 mg/kg NAC at t=−6 and t=0, 400-400 is 400 mg/kg NAC at t=−6 and t=0, and 400-0 is 400 mg/kg NAC at t=−6 and PBS at t=0 (** p<0.001). FIG. 6B: Clot stability for the repeat injection conditions (** p<0.001, * p<0.01). FIG. 6C: Bleeding times for mice treated with 400 mg/kg NAC vs PBS (* p<0.01).

FIG. 7 shows the effects of doses around 10 mM. Occlusion times for 7.5 mM, 20 mM and 40 mM doses tested in vitro in human blood. 7.5 mM dose has a mixed effect with ˜64% of samples not occluding within the time limit of the experiment. Both 20 mM and 40 mM doses did not occlude and had no visible platelet aggregation during the experiment, similar to the 10 mM condition (* p<0.05).

FIG. 8 shows the effect of WBC count on control OTs between different studies. Top: For individuals recruited in the current study, WBC counts above 6.3×10³/μL were correlated with lower occlusion times. Bottom: To test if this trend was present in data previously acquired in the same system, the untreated control group that was a part of the Aspirin study reported in a previous student's thesis was reviewed. The effect was also present in this data set, with WBC counts above 6.3×10³/μL having OTs of 128 s compared to 222 s for lower WBC counts (* p<0.05).

FIG. 9 shows platelet intensities following vWF replacement. Top: Average platelet intensities vs time for the control and 10 mM+19 IU vWF (recovery) conditions.

The first arrow marks the end of the lag phase for the control group and the last two arrows mark the occlusion times for both conditions. Bottom: Average platelet intensities for the recovery condition and the control shifted right by 77 s to emphasize the similar RPA rates.

FIG. 10 shows estimated NAC plasma concentrations of previous clinical trials. Table of estimated maximum concentration, average concentration, and concentration curve AUC for clinical trials in PCI/STEMI populations using a 3-compartment PK model.

FIG. 11 shows estimated plasma concentrations vs. time for efficacious doses used in vivo. Graph of estimated plasma concentrations vs. time for the current recommended treatment of acetaminophen OD and treatments similar to the in vitro and in vivo doses explored in this paper. Plasma concentrations were estimated using the 3-compartment pharmacokinetic model. The blue line represents a report of a fatal case from a NAC overdose.

FIG. 12 is a table of three compartment pharmacokinetic model parameters

### DETAILED DESCRIPTION

The present invention can be understood more readily by reference to the following detailed description of the invention and the Examples and Figures included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Further, the dates of publication provided herein can be different from the actual publication dates, which can require independent confirmation.

### Definitions

In this specification and in the claims that follow, reference will be made to many terms, which shall be defined to have the following meanings:

Throughout the present specification, numerical ranges are provided for certain quantities. It is to be understood that these ranges comprise all values and subranges therein. Thus, e.g., the range “from 1 to 10” includes all possible values therein (e.g., 1, 2, 3, 4, 5, 6, 7, 8, 9, and 10) and all possible ranges therein (e.g., 1-9, 2-8, 3-7, 4-6, 1-8, 2-7, 3-6, 4-5, 1-7, 2-6, 3-5, 1-6, 2-5, 3-4, etc.). Furthermore, all values within a given range may be an endpoint for the range encompassed thereby (e.g., the range 1-10 includes the ranges with endpoints such as 5-10, 6-10, etc.).

As used in the description and the appended claims, the singular forms “a,” “an,” and “the” include plural referents unless the context dictates otherwise. Thus, for example, reference to “a composition” includes mixtures of two or more such compositions, reference to “an inhibitor” includes mixtures of two or more such inhibitors and the like.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In some examples, a preparation can be administered therapeutically; that is, administered to treat an existing disease or condition. In some examples, a preparation can be administered prophylactically; that is, administered for prevention of a disease or condition. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with arterial thrombosis” means having been subjected to a physical examination by a person of skill, for example, a physician, and found to have a condition that can be diagnosed or treated by a compound or composition that can treat or prevent arterial thrombosis.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary and can be administered in one or more dose administrations daily, for one or several days. Guidance can be found in the literature for appropriate dosages for given classes of pharmaceutical products. In some examples, a preparation can be administered in a “prophylactically effective amount”; that is, an amount effective for prevention of a disease or condition.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to thrombosis) based upon an earlier diagnosis by a person of skill and thereafter subjected to treatment for the disorder. It is contemplated that the identification can, in some examples, be performed by a person different from the person making the diagnosis. It is also contemplated, in some examples, that the administration can be performed by one who subsequently performed the administration.

“Optional” or “optionally” means that the subsequently described event or circumstance can or cannot occur and that the description includes instances where the event or circumstance occurs and instances where it does not.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “reduce” or other forms of the word, such as “reducing” or “reduction,” it is meant lowering of an event or characteristic (e.g., thrombi formation). It is understood that this is typically in relation to some standard or expected value. In other words, it is relative, but it is not always necessary for the standard or relative value to be referred to. For example, “reduces thrombi formation” means decreasing the number of thrombi cells relative to a standard or a control.

As used herein, the term “subject” refers to the target of administration, e.g., patient. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, rodent, or fruit fly. The term does not denote a particular age or sex. Thus, adult and newborn subjects, as well as fetuses, whether male or female, are intended to be covered. In some examples, the subject is a mammal. A patient refers to a subject afflicted with a disease or disorder. The term “patient” includes human and veterinary subjects. In some examples of the disclosed methods, the subject has been diagnosed with a need for treatment or prevention of thrombosis.

The term “thiol” is represented herein by the formula --SH. Thus a compound containing a thiol is a compound with one or more --SH groups.

As used herein, the term “treatment” refers to the medical management of a patient with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder.

Reference will now be made in detail to specific aspects of the disclosed materials, compounds, compositions, articles, and methods, examples of which are illustrated in the accompanying Examples and Figures.

### Arterial Thrombosis

Disclosed are methods and compositions for treating and preventing arterial thrombosis, and reducing thrombi in arteries. Arterial thrombosis is the process which forms large, occlusive blood clot in arteries. These clots impede blood flow to downstream tissue, causing ischemic events such as heart attacks and strokes. Structurally, arterial thrombi are distinct from other clots such as venous thrombi and pulmonary emboli, which are comprised mainly of red blood cells (RBCs) and likely form through the coagulation pathway (Chernysh I N, et al. The distinctive structure and composition of arterial and venous thrombi and pulmonary emboli. Sci Rep. 2020;10(1):1-12). In contrast, arterial thrombi are made up of platelet-rich regions which can range from 11-99% of the total clot volume (Staessens S, et al. Structural analysis of ischemic stroke thrombi: Histological indications for therapy resistance. Haematologica. 2020;105(2):498-507). The formation of platelet-rich regions is the result of a significant process of concentrating platelets, given that platelets are ˜10× smaller and are at a ˜20× lower concentration compared to RBCs (Wiwanitkit V. et al. Mean Platelet Volume, Platelet Distribution Width: Its Expected Values and Correlation with Parallel Red Blood Cell Parameters. Clin Appl Thromb. 2004;10(2):175-178). This formation of platelet-rich regions can be explained by platelet margination to the wall of vessels and selective capture of platelets onto the growing mural thrombus (Aarts P A M M, et al. Blood platelets are concentrated near the wall and red blood cells, in the center in flowing blood. Arteriosclerosis. 1988;8(6):819-824).

Arterial thrombi are platelet rich because they form through Shear Induced Platelet Aggregation (SIPA) (Casa L D C, et al. Thrombus Formation at High Shear Rates. Annu Rev Biomed Eng. 2017;19(1):415-433). In regions of pathologically high shear stress, the plasma protein von Willebrand Factor (VWF) activates and can bind to both collagen and platelets near the vessel wall. Captured platelets form a new boundary that defines flow and release additional VWF, increasing the local VWF concentration by ˜50× and capturing additional nearby platelets. This kicks off a positive feedback loop which ends with the formation of a large, occlusive clot (Kim D, et al. Occlusive thrombosis in arteries. APL Bioeng. 2019;3(4)).

The disclosed compositions and methods address the problem of arterial thrombosis from a different point-of-view than to concentrate on the inhibition of VWF. There is little published work suggesting inhibition of the linker protein, VWF, prevents occlusive thrombosis in major arteries.

Some approaches involve the removal of VWF, cleavage of VWF by mechanical devices such as ECMO or artificial heart valves, or antibodies against the Al domain (Geisen U, et al. Non-surgical bleeding in patients with ventricular assist devices could be explained by acquired von Willebrand disease. Eur J Cardio-Thoracic Surg. 2008;33:679-84; Li B X, et al. In vitro assessment and phase I randomized clinical trial of anfibatide a snake venom derived anti-thrombotic agent targeting human platelet GPIba. Sci Rep. 2021;11(1):1-17; Scully M, et al. Caplacizumab Treatment for Acquired Thrombotic Thrombocytopenia Purpura. N Engl J Med. 2019;380(4):335-467-9). Others have suggested attacking VWF for treatment of Thrombotic Thrombocytopenia Purpura, a microvessel disease that does not affect larger arteries (Chen J, et al. N-Acetylcysteine Treatment in Two Patients with Relapsed Thrombotic Thrombocytopeniaurpura Increased ADAMTS13 Activity, Free Thiol Concentration in Plasma, and Inhibited Platelet Activation. Blood. 2015;126(23):239-239).

Targeting vWF instead of platelets is an alternative mechanism for reducing arterial thrombosis. The most straight forward way of targeting vWF is to prevent vWF from binding to platelets using molecules that competitively bind either platelets or vWF. Several groups have developed drugs to achieve this and are starting the lengthy process of showing safety and efficacy in human subjects (Li B X, Id.; Scully M, Id.; Nimjee S M, et al. Preclinical Development of a vWF Aptamer to Limit Thrombosis and Engender Arterial Recanalization of Occluded Vessels. Mol Ther. 2019;27(7):1228-41).

Thus, disclosed herein are methods of treating or preventing arterial thrombosis in a subject in need thereof comprising administering to the subject a thiol containing compound. Methods of preventing arterial thrombi formation, e.g., shear induced platelet aggregation, comprise administering to the artery a thiol containing compound.

In the disclosed methods, the arterial thrombus being treated, prevented or reduced is a blood clot, e.g., an aggregation of certain components, such as platelets and/or fibrin, formed, for example, in response either to an atherosclerotic lesion or to vessel or tissue injury. In certain examples, the thrombus is a white thrombus that is characterized by a predominance of platelets and/or von Willebrand Factor (VWF), and, in some cases, a paucity of red blood cells. In certain examples, the thrombus is substantially free of red blood cells. In some examples, the thrombus has a concentration of red blood cells of less than about 30%, or less than 25%, or less than 20% or less than 15% or less than 10%, or less than about 5%, or less than about 1%, or less than 0.5%,

Further, selenium containing compounds can be used as an alternative, or in addition to the thiol containing compounds in the disclosed methods. A suitable subject can be one undergoing angioplasty or percutaneous coronary intervention, that has a heart attack, or that has ischemic stroke. Subjects that are at high risk of forming arterial thrombi can also benefit by the disclosed compositions and methods. Determining whether the subject is at high risk of forming arterial thrombi can be ascertained by measuring the subject's blood concentration levels of vWF. For example, if the blood concentration of vWF is in an upper quartile, the subject can be at high risk of forming arterial thrombi.

### Thiol Containing Compounds

As VWF has many disulfide bonds, the approach disclosed herein disrupts these disulfide bonds to lead to a loss of function of VWF in forming platelet-rich thrombi (Solecka B A, et al. Free thiol groups in von Willebrand factor (VWF) are required for its full function under physiological flow conditions. Thromb Res. 2016;137:202-10). Reducing the length of vWF also decreases its platelet binding affinity. Compounds suitable for use in the disclosed methods are thus thiol containing compounds that can disrupt the disulfide bonds of vWF. In specific examples, the thiol containing compounds that can be used herein can have on or more (e.g., 2, 3, or 4) free thiol groups.

N-acetylcysteine (NAC) is disclosed herein as one example for use in targeting vWF and reducing the formation of arterial thrombi. NAC is a reactive free-thiol that can break existing disulfide bonds through disulfide exchange (Samuni Y, et al. The chemistry and biological activities of N-acetylcysteine. Biochim Biophys Acta-Gen Subj. 2013;1830(8):4117-4129). Previous research focused on using NAC as a thrombolytic agent based on structural similarities between vWF and mucins; they are both large proteins polymerized by disulfide bonds (Ferez-Vilar J, et al. The structure and assembly of secreted mucins. J Biol Chem. 1999;274(45):31751-31754; Chen J, et al. N-acetylcysteine reduces the size and activity of von Willebrand factor in human plasma and mice. J Clin Invest. 2011;121(2):593-603). In treating cystic fibrosis, NAC is inhaled as a mist and cleaves the disulfide bonds, shortening mucins and loosening mucus (Webb W R. Clinical evaluaton of a new mucolytic agent, acetyl-cysteine. J Thorac Cardiovasc Surg. 1962;44(3):330-343). A similar chemical mechanism was proposed for vWF where NAC would lyse clots by cleaving vWF (Chen J, et al. J Clin Invest. 2011;121(2):593-603). In support of this, NAC has been shown to reduce the size of plasma vWF and decrease the activity of vWF. NAC has also been shown to reduce a key disulfide bond linking Cys1272 and Cys1458 across the A1 domain (Id.). Given the importance of disulfide bonds in creating secondary structure of the Al platelet binding domain, NAC has multiple potential mechanisms for reducing vWF activity (Solecka B A, et al. Free thiol groups in von Willebrand factor (VWF) are required for its full function under physiological flow conditions. Thromb Res. 2016;137:202-210).

The previous work investigated NAC's ability to cleave vWF and focused on lysing existing clots with the goal of resolving TTP acute events (Chen J, et al. N-Acetylcysteine Treatment in Two Patients with Relapsed Thrombotic Thrombocytopeniaurpura Increased ADAMTS13 Activity, Free Thiol Concentration in Plasma, and Inhibited Platelet Activation. Blood. 2015;126(23):239-239). These experiments are complicated by the challenge of delivering NAC to an occluded artery where flow is minimal. In this work, it was hypothesized that treatment of vWF with NAC will affect SIPA formation and growth rate. This work focused on using NAC for prevention of thrombosis instead of lysis. In one microfluidic model, NAC has been shown to prevent platelets from aggregating, but the initial and maximum shear rates in that model were relatively low at 0 and 2000 s⁻¹, respectively (Herbig B A, et al. Thrombi Produced in Stagnation Point Flows Have a Core-Shell Structure. Cell Mol Bioeng. 2017;10(6):515-521). This model was not designed to form occlusive thrombi under high arterial shear conditions but rather to grow from a stagnant region; therefore, it remains unclear if these results will apply to arterial SIPA thrombosis. Another previous study investigated NAC for prevention of arterial thrombosis in vivo but failed to prevent clot formation (Martinez de Lizarrondo S, et al. Potent Thrombolytic Effect of N-Acetylcysteine on Arterial Thrombi. Circulation. 2017;136:646-660). This is likely due to their use of the FeCl injury model; subsequent work has shown that this model leads to the formation of large colloidal gels that are not representative of arterial thrombosis (Ciciliano J C, et al. Resolving the multifaceted mechanisms of the ferric chloride thrombosis model using an interdisciplinary microfluidic approach. Blood. 2015;126(6):817-824.) This study was performed in a modified-Folts model in mice. This model creates an artificial stenosis with high shear rates and exposes subendothelial collagen in the carotid artery with a crush injury to generate thrombi that are platelet-rich and representative of SIPA (Kim D A, et al. Platelet α-granules are required for occlusive high-shear-rate thrombosis. Blood Adv. 2020;4(14):3258-3267).

Additional thiol containing compounds that can be used herein are cysteine (either D or L or a mixture thereof), dithiothreitol, glutathione, dimercaptosuccinic acid, thioterpinol, methanethiol, ethanethiol, or any combination thereof. In other examples, the thiol containing compound can be a peptide containing cysteine, e.g., a peptide with 2, 3, 4, 5, 6, 7, 8, 9, or 10 amino acids containing at least one cysteine residue.

In other examples, the thiol containing compound can be the disulfide dimer of cysteine known as cystine, or the mono-N-acetyl cystine or di-N-acetylcystine (diNAC), including any optical isomer thereof. In other examples, cystine, mono-N-acetyl cystine, and di-N-acetylcystine (diNAC), alone or in any combination, can be combined with N-acetyl cysteine, cysteine, dithiothreitol, glutathione, dimercaptosuccinic acid, thioterpinol, methanethiol, ethanethiol, or any combination thereof.

### Selenium Containing Compounds

As an alternative, or in addition to, the thiol containing compounds, selenium containing compounds can be used in the disclosed methods. An example of suitable selenium compounds that can be used is selenocysteine.

### Administration

The disclosed compounds can be administered sequentially or simultaneously in separate or combined pharmaceutical formulations. When one or more of the disclosed compounds is combined with a second therapeutic agent, the dose of each compound can be either the same or differ from that when the compound is used alone. Appropriate doses will be readily appreciated by those skilled in the art.

In vivo application of the disclosed compounds and compositions containing them can be accomplished by any suitable method and technique presently or prospectively known to those skilled in the art. For example, the disclosed compounds can be formulated in a physiologically-or pharmaceutically-acceptable form and administered by any suitable route known in the art, including oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration or at continuous or distinct intervals as readily determined by a person skilled in the art.

The compounds disclosed herein and compositions comprising them can also be administered utilizing liposome technology, slow-release capsules, implantable pumps, and biodegradable containers. These delivery methods can provide a uniform dosage over an extended period. The compounds can also be administered in their salt derivative forms or crystalline forms.

The compounds disclosed herein can be formulated according to known methods for preparing pharmaceutically acceptable compositions. Formulations are described in detail in many sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier to facilitate the effective administration of the compound. The compositions used can also be in a variety of forms. These include, for example, solid, semisolid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired treatment, compositions disclosed herein can advantageously comprise between about 0.1% and 99%, and especially, 1 and 15% by weight of the total of one or more of the subject compounds based on the weight of the total composition including carrier or diluent. Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example, sealed ampoules and vials. The formulations can be stored in a freeze-dried (lyophilized) condition requiring only the condition of the sterile liquid carrier, for example, water for injections, before use. Extemporaneous injection solutions and suspensions can be prepared from sterile powder, granules, tablets, etc. It should be understood that in addition to the ingredients particularly mentioned above, the compositions disclosed herein can include other agents conventional in the art regarding the type of formulation in question.

Compounds disclosed herein and compositions comprising them can be delivered to a cell either through direct contact with the cell or via a carrier. Carriers for delivering compounds and compositions to cells are known in the art and include, for example, encapsulating the composition in a liposome moiety.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, for example, by the formation of liposomes, by the maintenance of the required particle size in the case of dispersions, or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by including agents that delay absorption, such as aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

In some embodiments of the disclosed treatment methods, the subject can be administered a dose of thiol containing compound (or selenium containing compound) as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg). In some examples, the subject is administered a single dose of thiol containing compound (or selenium containing compound).

In some embodiments of the disclosed treatment methods, the subject can be administered a dose of thiol containing compound (or selenium containing compound) sufficient to result in a concentration of the compound in the subject's blood of up to 5 mM, e.g., up to 1 mM, 1.5 mM, 2 mM, 2.5 mM, 3 mM, 3.5 mM, 4 mM, 4.5 mM or 5 mM, where any of the stated values can form an upper or lower endpoint of a range. In other examples, the subject can be administered a dose of thiol containing compound (or selenium containing compound) sufficient to result in a concentration of the compound in the subject's blood of up to 10 mM, e.g., up to 5.5 mM, 6 mM, 6.5 mM, 7 mM, 7.5 mM, 8 mM, 8.5 mM, 9 mM, 9.5mM, or 10 mM, wherein any of the stated values can form an upper or lower endpoint of a range.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

N-acetylcysteine was initially assessed for its ability to prevent formation of arterial thrombi by measuring if it significantly reduces platelet accumulation rates and increases occlusion times in a microfluidic model of arterial thrombosis using human blood. Clot formation and stability was then measured in the acute murine modified-Folts model to verify the in vitro results with an in vivo model and to see if there are any unintentional side effects, such as increased bleeding. The effect of NAC in the murine model after 6 hrs. was measured to determine if the effects persist after NAC has been cleared from the blood.

Microfluidic tests were run using a microfluidic system previously described (Griffin M T, et al. Inhibition of high shear arterial thrombosis by charged nanoparticles. Biomicrofluidics 2018;12). The chip contains a single inlet split into four outlet channels 475 μm wide and 250 μm tall. Within each outlet channel is a stenotic region 800 μm long where the height decreases to 70 μm. Flow is controlled using a hydraulic pressure head created by a raised reservoir connected to the inlet which generates an initial shear rate of ˜7,500 s-1 in the stenotic region. Channels are coated overnight with 100 μg/mL collagen Type I (Chrono-Log Corp) in 0.9% saline. Blood is filtered with a 37 μm nylon mesh immediately prior to testing to prevent debris from entering the chip.

For each chip, the mass flow rate of each outlet channel and brightfield images of two stenotic regions were acquired. Mass flow rates for each channel were measured using four mass balances (Ohaus Scout) connected to the device outlets. Occlusion time was defined as the difference between the time blood enters the stenosis region of the microfluidic device and the time when the mass balances register their peak mass. A brightfield, upright microscope (Leica DM6000B) was used in conjunction with a CCD camera (Pixelfly, PCO) to image the stenotic regions of the middle two channels as the occlusive thrombus formed. Images were taken once per second with a 40× magnification. These images were later thresholded into black and white images to identify platelet rich thrombi from the background blood (ImageJ). Average platelet intensity is defined as the average B/W value of the stenosis region. A value of 0 represents a clean channel with no platelets and increasing values indicate platelet accumulation.

Occlusion times for each concentration were tested using an unpaired Student's t-test. For survival curves, each possible combinations of pairs of concentrations were tested using a log-rank test. Visual measurement of platelet intensities for each concentration were compared using a Mann-Whitney U test. Statistical significance was determined by p<0.05.

### In Vitro Blood Collection and Sample Preparation

Blood was collected from 10 healthy adult volunteers. Subjects were recruited in accordance with Georgia Institute of Technology's Institutional Review Board Guidelines (IRB #17315). Subjects with known bleeding disorders, a history of anti-platelet medications within 10 days of donation, anemia or transmittable blood disease were excluded from the study. Standard phlebotomy techniques were used to obtain blood. Briefly, 60 mL of blood was drawn slowly through a 21 G needle into a 60 ml syringe containing 0.21 mL of a 1000 USP/mL solution of heparin sodium (Fisher Scientific) in PBS to achieve a heparin concentration of 3.5 USP/L in the collected blood. Complete Blood Counts (CBCs) were also performed on all donor samples. All tests were run within 4 hours of the blood draw.

N-acetyl cysteine was added to blood to create final concentrations of 3 mM, 5 mM and 10 mM. For each human subject, a fresh solution of concentrated NAC (Sigma Aldrich) was created in PBS and neutralized to a pH of 7 with NaOH. The concentrated NAC solution was then diluted so that the same volume of NAC solutions was added to each sample. Negative control (OmM) samples were similarly diluted with 0.5 mL of PBS in 15 mL blood. In three individuals, additional experiments were performed at concentrations of 7.5 mM, 20 mM and 40 mM. For all experiments, NAC was allowed to incubate in the blood at room temperature on a shaker for 30 minutes prior to testing.

VWF recovery experiments were performed using 3 individual's 10 mM NAC samples prepped as detailed above. 19 IU/mL of VWF (Humate) was added to these samples and they were immediately perfused through the microfluidic system. Occlusion times and platelet intensities were measured as above.

### In Vivo Model of Arterial Thrombosis

Formation of occlusive thrombi was measured using a murine Modified-Folts model (Kim D A, et al. Platelet α-granules are required for occlusive high-shear-rate thrombosis. Blood Adv. 2020;4:3258-3267). C57 mice were used under conditions approved by the university animal use and care committees. Following isolation of the common carotid artery, blood flow through the vessel was measured with a flow probe (Transonic Systems). A 6.0 silk suture was then tied around the vessel and tightened until the measured flow rate was approximately 50% of the initial value. This creates a stenosis region with high shear-rate at the apex. The artery is then crushed with forceps to damage the endothelial cells and expose collagen in the extracellular matrix.

In single injection experiments, 13 mice were treated with either 200 mg/kg NAC, 400 mg/kg NAC, or PBS. 100 μL of freshly prepared, neutralized NAC solution or vehicle was I.V. injected approximately 45 minutes prior to the crush injury.

For repeat injection experiments, mice were given either PBS, 200 mg/kg, or 400 mg/kg NAC through a 100 μL I.V. injection 6 hr prior to surgery. Mice were then given a second injection of PBS, 200 mg/kg, or 400 mg/kg NAC approximately 45 minutes prior to the crush injury. Additionally, 4 mice were given a dose of 400 mg/kg NAC through an 100 μL injection 6 hr prior to the surgery, followed by a second injection of PBS immediately prior to the crush injury.

Blood flow measurements were taken with a Transonic flow probe and acquired with Powerlab software (ADInstruments) (Id.). Time to initial occlusion is defined as the time between the crush injury and the first point where flow reaches approximately 0. After occlusion, if flow remains around 0 then the occlusion is characterized as stable. If instead the thrombus detaches and flow increases, the clot is labelled an unstable occlusion.

Bleeding times were assessed using the tail bleeding model described previously (Liu Y, Standardizing a simpler, more sensitive and accurate tail bleeding assay in mice. World J Exp Med. 2012;2:30). Briefly, mice were anesthetized and the distal 3 mm segment of the tail was transected and placed in a vial of warm saline. The injuries were monitored visually to determine the time it took for bleeding to cease.

Statistical significance in time to initial occlusion was determined using unpaired student t-test (p <0.05). Differences in thrombus stability are determined using a Pearson's Chi-squared test (p <0.05).

### Pharmacokinetic Modeling

A 3-compartment PK model was created in compliance with the guidelines published by A. Tavlei et. al, The ADME Encyclopedia 2021. Literature values utilized for the model are listed in FIG. 12 (Teder K, et al. The pharmacokinetic profile and bioavailability of enteral n-acetylcysteine in intensive care unit. Med. 2021;57:1-12). For comparsion, dosing schedules for 14 clinical trials using NAC and reporting MACE were included, as well as the standard guidelines for NAC treatment following acetominophen overdose and a case study describing a fatal overdose of NAC.

### NAC Increases Occlusion Time in a Dose-Dependent Manner in Human Blood

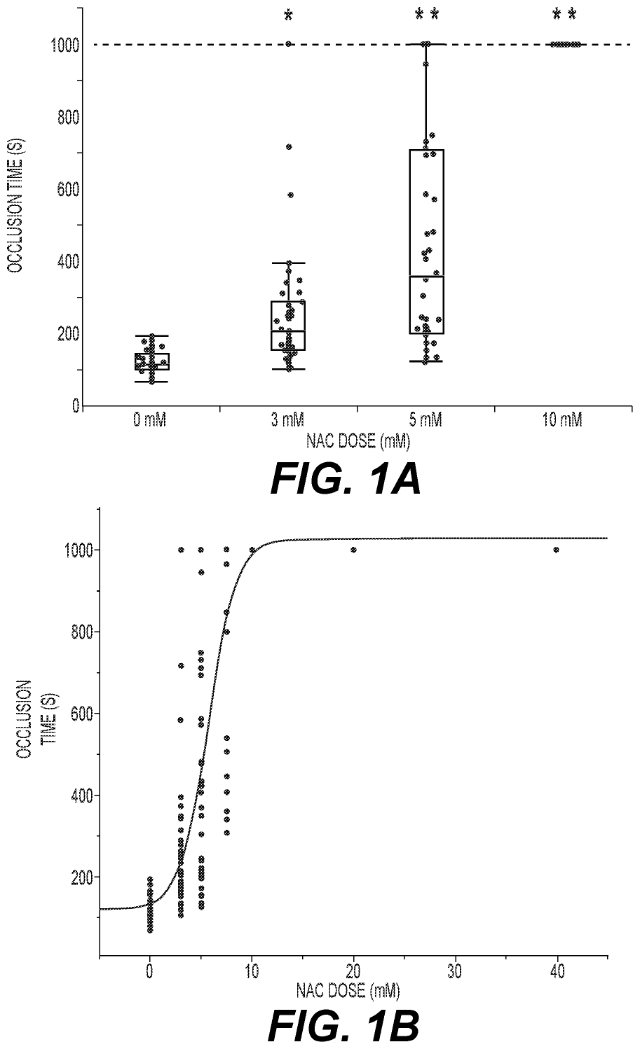

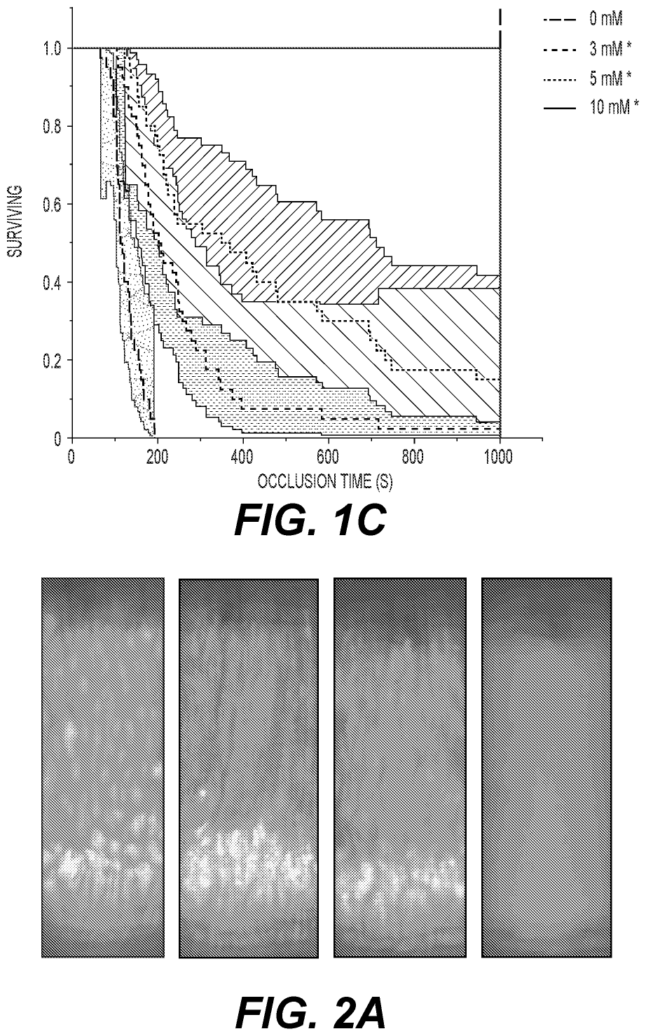

To test the effect of N-acetylcysteine on reducing arterial thrombosis in human blood, whole blood was perfused collected from 10 healthy human subjects through a stenotic microfluidic channel coated with type I collagen. A constant pressure head of 16 mmHg was formed using an elevated upstream reservoir to create shear rates of ˜7,500 s-1 in the stenotic region and ˜800 s-1 in the rest of the channel. Untreated blood formed stable occlusive thrombi in 124 +/−30 s. (FIG. 1A) Average occlusion times for blood treated with NAC were 255, 458 and 1000s for 3 mM, 5 mM and 10 mM doses respectively (p<0.01 ). Experiments were terminated at 1000 s, with an occlusion time value of 1000 s indicating that occlusion did not occur during the experiment. A 4-parameter logistic fit of the data has a lower asymptote at 118.5 s, an upper asymptote at 1028.4 s, a logistic growth rate of 0.7145 and an inflection point at 5.6545 mM NAC. This fit gives an r² value of 0.782 (FIG. 1B). Survival curves were generated for each dose where survival indicates a channel remained unoccluded. Survival rates increased with NAC concentration. The survival curves generated for each dose are all statistically significantly different from each other (p<0.001) (FIG. 1C).

### High Concentrations of NAC Completely Prevent Platelet Aggregation

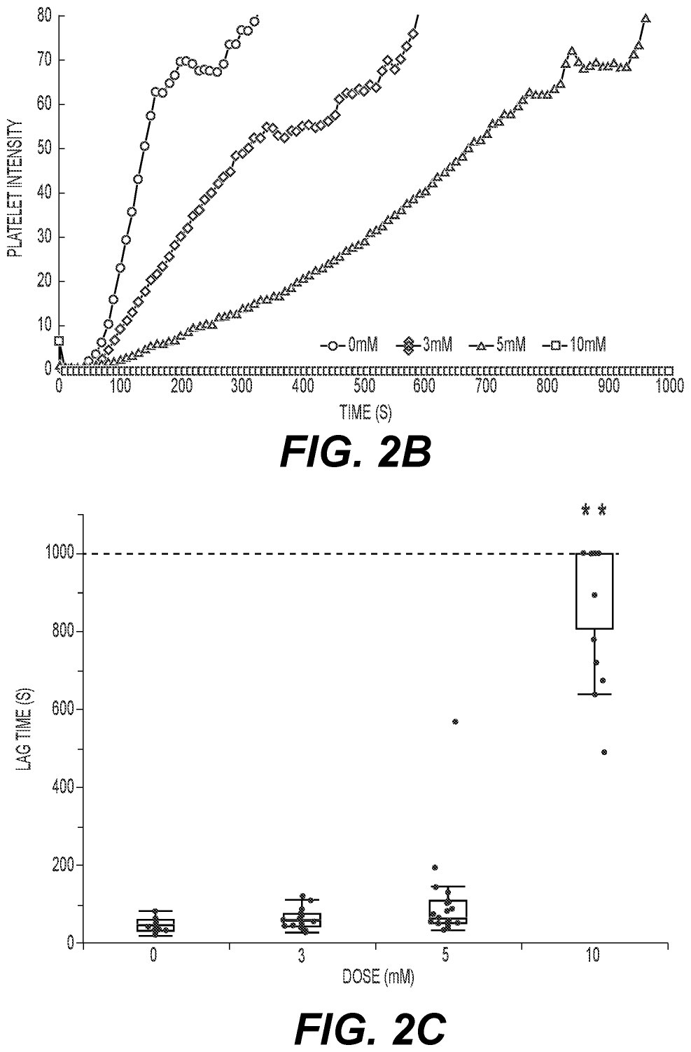

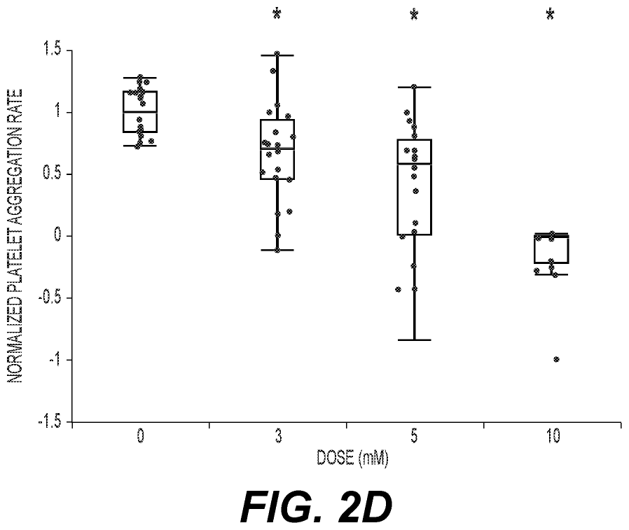

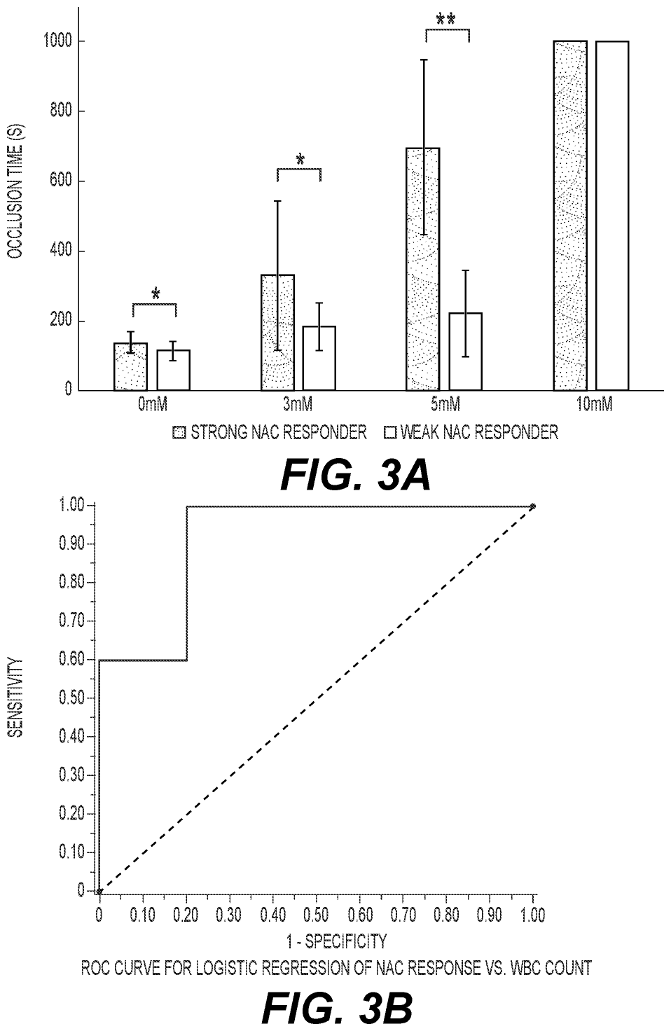

Brightfield videos of the middle two channels of the microfluidic device were used to determine whether 10 mM NAC was completely preventing platelet aggregation or lengthening occlusion times beyond 1000 s. In the images of 10 mM NAC, no visible aggregation of platelets is observed during the 1000 s of the experiment (FIG. 2A). In comparison, platelet aggregates were visible in the control and low dose NAC cases starting around 35-60 s. At these lower doses of NAC the occlusive thrombus was located near the entrance of the stenotic region, as previously described (FIG. 2A).

Clot formation abruptly changes from reduced accumulation to no accumulation as the concentration of NAC rises from 5 mM to 10 mM. To check if this effect occurs between 5 mM and 10 mM and if it persists beyond 10 mM, additional experiments at 7.5 mM, 20 mM and 40 mM were performed on 3 individuals. Concentrations over 10 mM show no visible platelet aggregation, suggesting the 10 mM results can be extrapolated for any concentration over 10 mM. For the 7.5 mM dose, results were mixed with some channels occluding within the 1000 s window and others not occluding during the experimental window (FIG. 7).

### Lengthening of Occlusion Time Correspond with a Decrease in Platelet Aggregation Rate

Brightfield measurements of platelet aggregation were used to establish whether the increase in occlusion time observed in 3 mM and 5 mM cases is primarily due to effects on initial platelet attachment (lag time) or the platelet accumulation rate (RPA). Lag time is defined as the time required for the average platelet intensity measurements to increase to 1% of their maximal value. Average lag times for 0 mM, 3 mM, 5 mM NAC experiments are 49 s, 65 s and 107 s, respectively. The small increases in lag time for 3 mM and 5 mM treatments cannot account for the 125-325 s increases in occlusion time seen in the 3 mM and 5 mM conditions. This is instead explained by a decrease in the rapid platelet accumulation rate, defined as the slope of the platelet intensity graph (FIG. 2B). The lag time for 10 mM NAC is significantly higher at 910 s (FIG. 2C, p<0.0001). The slope of the platelet intensity curve decreases as NAC concentration increases, with 10 mM NAC having a negligible slope. When the RPA rate for each individual is normalized to the individual's control RPA rate, the platelet aggregation rates for 3 mM, 5 mM and 10 mM are 0.66, 0.38 and −0.12, respectively (FIG. 2D, p<0.01 compared to control for all conditions). The small negative aggregation rate for 10 mM is an artifact caused by mixing of blood and PBS at the interface between the priming solution and the sample. The magnitude of RPA rates differed between individuals, and some individuals had a stronger response to low doses of

NAC, but the trend of 3 mM and 5 mM having lower RPA rates compared to OmM and 10 mM having the lowest RPA rate was consistent between individuals. (p<0.05 Wilcoxon Each Pair).

### Strong NAC Response Corresponds with Low WBC Count

While 3 mM NAC caused a slight increase in OT for almost all subjects, in half of the subjects there was no additional increase in occlusion time when the concentration of NAC was increased from 3 mM to 5 mM. The population was split into two groups labelled strong NAC responders or weak NAC responders with strong NAC responder's blood having a statistically significantly higher occlusion time when treated with 5 mM vs. 3 mM NAC. After making this distinction, the CBC data was reviewed to determine if any factors such as platelet count or hematocrit could explain the variability observed between individuals. Surprisingly, the strongest predictor of whether an individual would be classified as a strong NAC responder was the WBC count. (Prob>Chi²=0.0079) Individuals with WBC counts under 6.3×10³/μL were more likely to be strong NAC responders than those with WBC counts over that value. (AUC 0.92) It was then considered whether strong

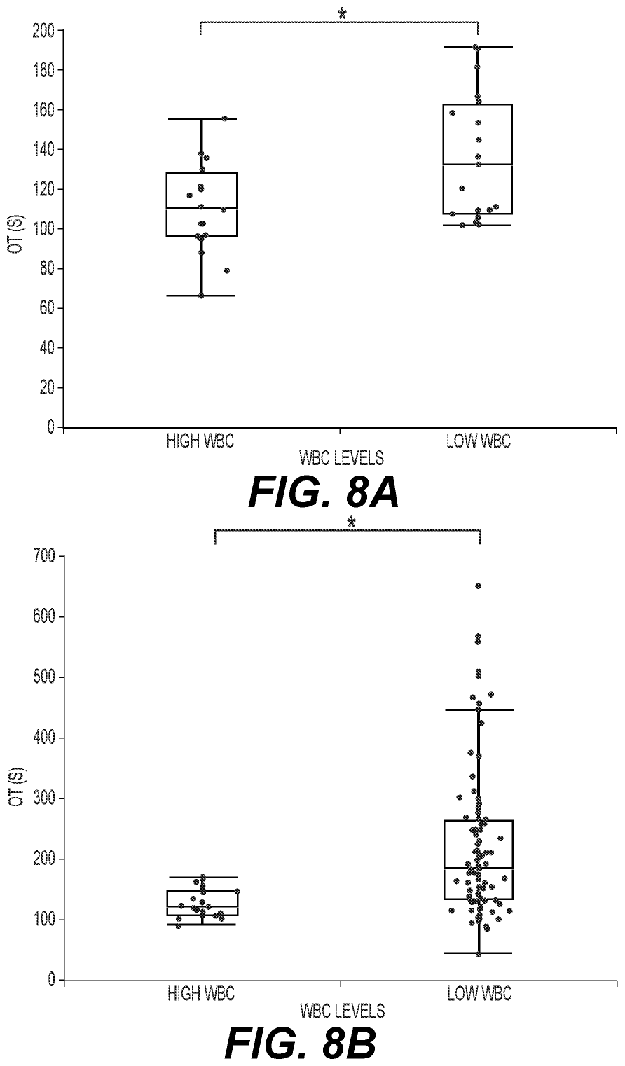

NAC responders had different OT compared to weak NAC responders. In the control, 3 mM and 5 mM populations, strong NAC responders had significantly higher OTs compared to those weak NAC responders. (p<0.01) Occlusion times for the weak NAC responders were 112 s, 182, and 220 s for OmM, 3 mM, and 5 mM respectively. Occlusion times for the strong NAC responders were 136 s, 328 s and 695 s for OmM, 3 mM, and 5 mM respectively. This correlation between WBC count and control OT was also observed in data previously published using the same microfluidic chip for a different study (FIG. 8).

### Recovering Thrombus Formation with Addition of VWF

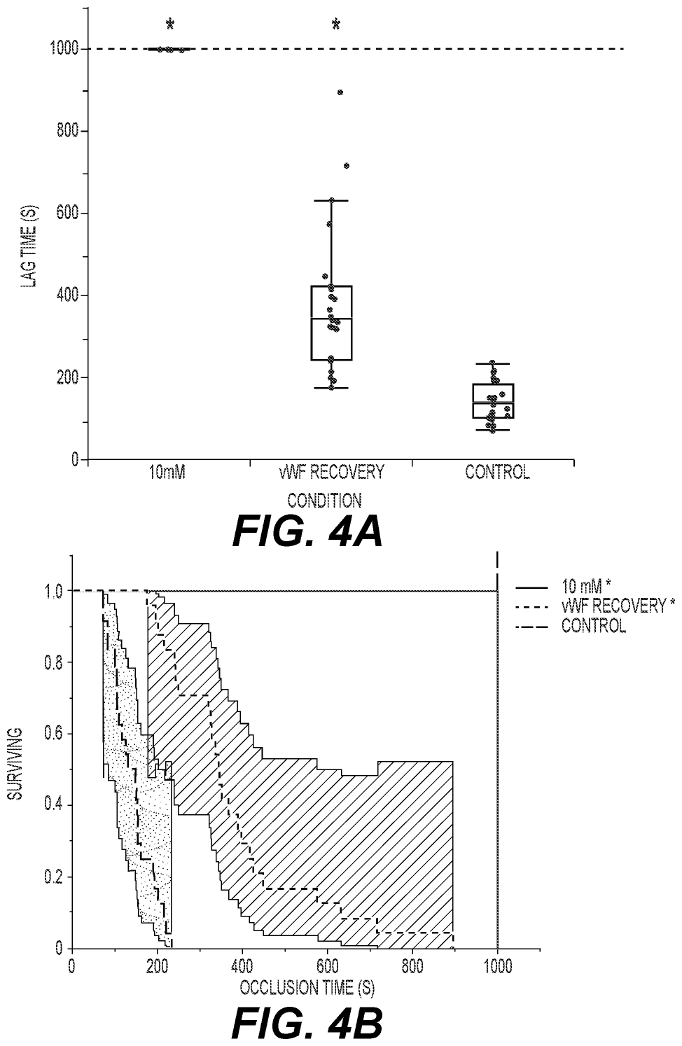

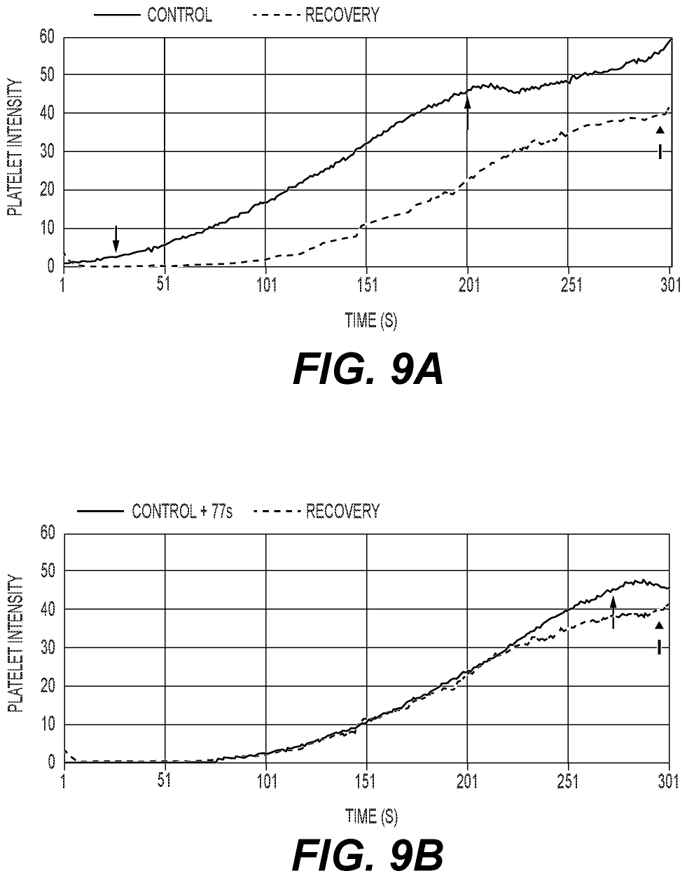

To test if the NAC prevented arterial thrombosis by modifying VWF as hypothesized, new VWF was added to blood samples treated with 10 mM NAC. The added VWF allowed the blood to recover the ability to form rapid occlusive thrombi (FIG. 4, p<0.001). Although OTs of the recovery samples were higher than the control, the average RPA rates for the control and recovery conditions were similar. By shifting the control curve right, simulating a 76 s increase in lag time, the two curves show a high degree of concordance (FIG. 9).

### Administration of NAC Prior to Vascular Injury Reduces Arterial Thrombosis In Vivo

In the murine modified Folts model, average initial occlusion times measured with the Transonic flow probe increased from 4.6 to 16.6 minutes when a 200 mg/kg NAC (N=4) was given immediately prior to the crush injury compared to the vehicle injection (N=5) (p=0.16). For mice given 400 mg/kg NAC (N=3), no occlusion was observed, and flow rates remained steady during the 45 minutes of the experiment (p<0.01). A lower basal blood flow and initial labored breathing was observed following administration of NAC.

### Clots Formed with Low Doses of NAC are Less Stable than Control Clots

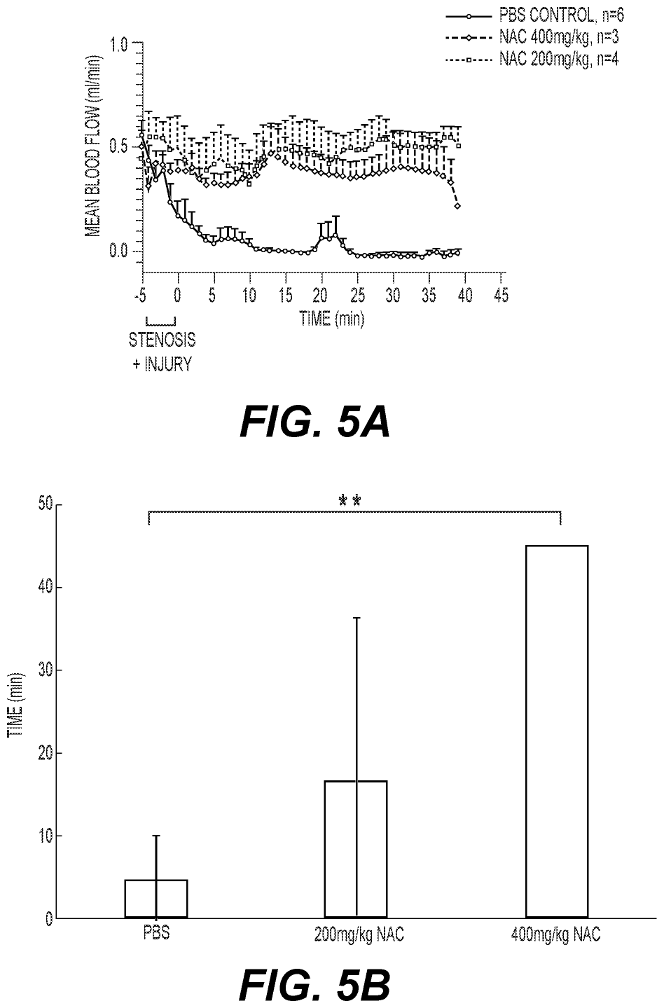

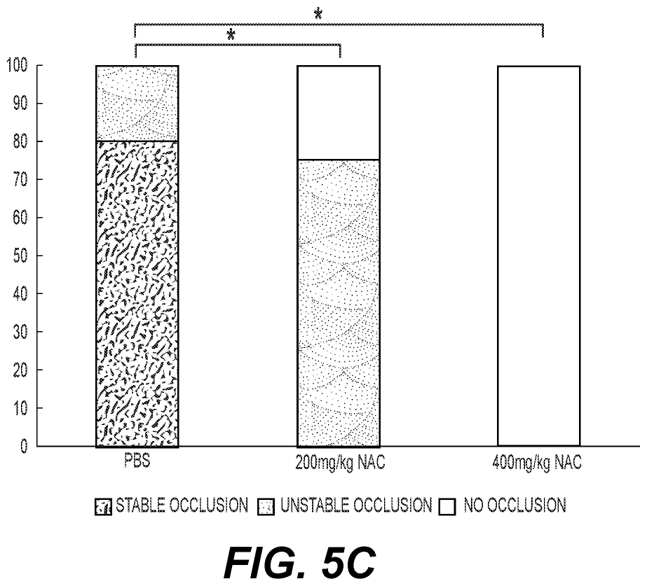

The majority of clots formed following injection of PBS remained occlusive during the entirety of the experiment. In one of the mice with receiving PBS, flow increased 8 minutes after the initial occlusion, indicating thrombus embolization. However, this artery re-occluded within 6 minutes and remained occluded. In comparison, all clots formed after injection of 200 mg/kg NAC recanalized completely after initially occluding, returning to ˜100% of the initial flow (FIG. 5A-5C, p<0.05).

### Repeat Administration of Low Dose NAC

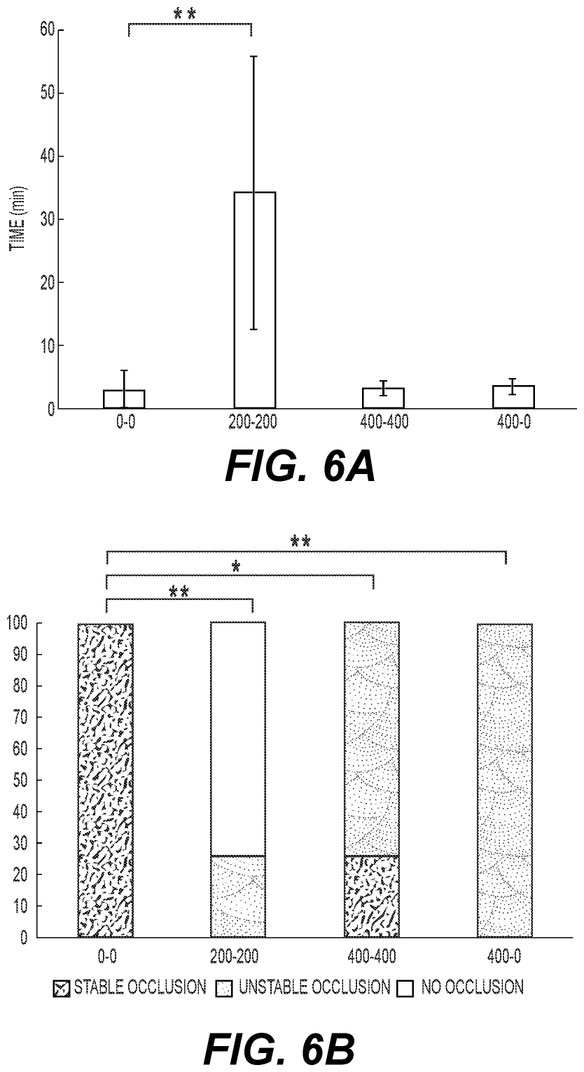

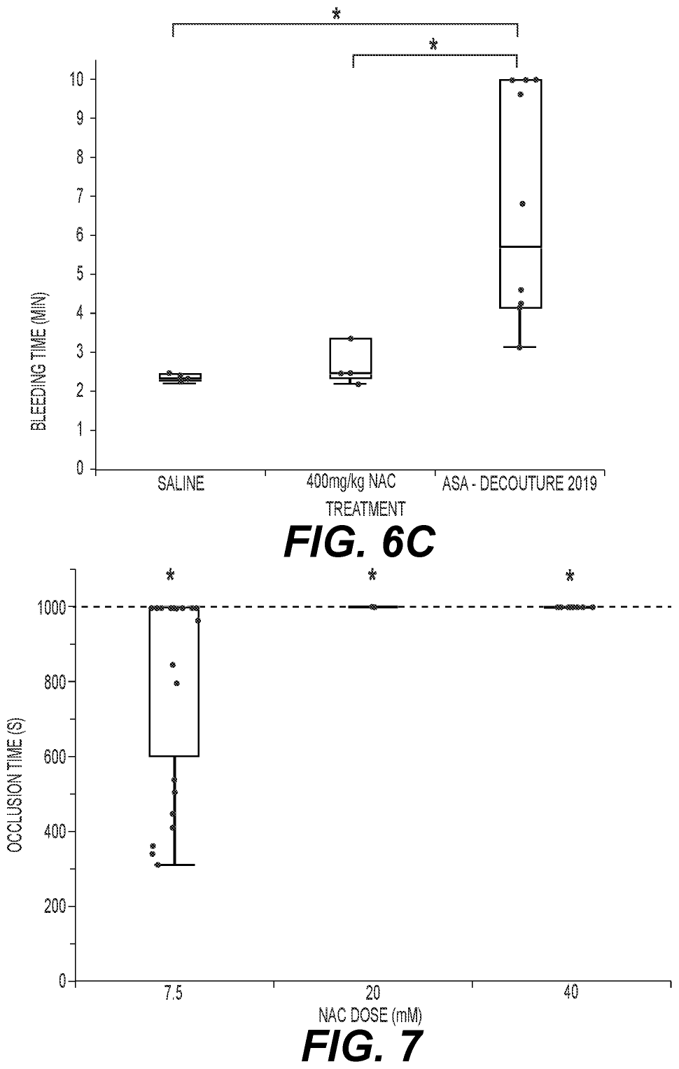

All mice given two injections of PBS spaced 6 hrs. apart formed occlusive, stable thrombi, similar to the results after a single injection of PBS. Three of the 4 mice given 200 mg/kg doses 6 hrs prior and immediately prior to crush injury formed no thrombi and remained patent during the entire experiment. The other mouse formed an initial thrombus that was quickly resolved and did not clot again for the remainder of the experiment. Unexpectedly, the mice given two doses of 400 mg/kg NAC all clotted within 10 minutes, with the majority forming unstable thrombi (FIGS. 6A-6B). The tissue at the crush injury site for these mice was observed to be significantly more delicate than all other conditions and the crush injury led to more severe tissue damage and hemorrhage. In two mice of the mice given 2 injections of 400 mg/kg NAC, the experiments were terminated early due to excessive blood loss at the injury site which was impeding blood flow measurements.

### Effects of NAC-Mediated Prevention of Arterial Thrombosis Persist for up to 6 hrs. in the Modified Folts Model

Three mice were given 400 mg/kg 6 hrs. prior to surgery followed by an injection of PBS immediately prior to crush injury to determine if NAC has lasting effects on arterial thrombosis after being eliminated from the plasma. Given an estimated elimination half-life in mouse plasma of 34 minutes, the plasma concentration of NAC at 6hrs. should be <0.05% of the initial concentration (Zhou J, et al. Intravenous Administration of Stable-Labeled N-Acetylcysteine Demonstrates an Indirect Mechanism for Boosting Glutathione and Improving Redox Status. J Pharm Sci. 2015;104:2619-2626). The mice that received 400 mg/kg NAC 6hrs prior to surgery formed unstable thrombi. Occlusion times in these mice were similar to control mice but like the single injection of 200 mg/kg NAC, the vessels treated with 400 mg/kg NAC recanalized and remained patent (FIGS. 6A and 6B). Thus, NAC has persistent effects on preventing arterial thrombosis in vivo. This result would be consistent with a long-lasting or permanent effect on the VWF molecule.

### Tail Bleeding

Mice treated with 400 mg/kg NAC had bleeding times of an average 2.76 minutes, not significantly different from mice treated with PBS, which had bleeding times of average 2.35 minutes (FIG. 6C, p=0.14). For comparison, this is much lower than the average of 5.72 minutes previously seen in mice treated with ASA (p<0.01) (Decouture B, et al. Evaluation of commonly used tests to measure the effect of single-dose aspirin on mouse hemostasis. Prostaglandins Leukot Essent Fat Acids 2019;149:46-51).

### Discussion

This study investigated the ability of NAC to inhibit platelet aggregation and prevent arterial thrombosis. This is the first study to look at the effects of NAC on the formation of large, occlusive thrombi and to observe complete prevention of arterial thrombosis with NAC in both in vitro and in vivo models. By choosing to use the stenotic modified-Folts model, the formation of colloidal gels that can occur in the ferric chloride model and complicate measurement of platelet aggregation were avoided. These in vitro experiments reveal NAC has a dose-dependent effect on platelet aggregation rate reducing the maximum growth rate of thrombi by 30-60% for 3 mM and 5 mM concentrations, respectively. No platelet aggregates were observed when NAC plasma concentrations are 10 mM, but the addition of 19 IU VWF led to recovery of the ability of blood to form occlusive thrombi.

The ability of NAC to prevent platelet thrombi is also demonstrated in mice treated with 400 mg/kg NAC that formed no clots. Reduced clot stability with the addition NAC was also observed; clots formed in mice treated with either dose were significantly more likely to spontaneously recanalize than mice treated with a saline injection.

These observations on the association between low WBC counts with an increased NAC response and higher control occlusion times were unexpected. While not wishing to be bound by theory, it is believed that WBC count is a marker of inflammation linked with changes in VWF. In addition to inflammation causing additional release of VWF from endothelial cells, previous research has shown that inflammation is associated with especially active VWF. The presence of VWF with increased activity in individuals with high WBC counts could lead to shorter occlusion times and require more NAC to modify the VWF to a level that completely prevents arterial thrombosis (Hyseni A, et al. Active von Willebrand factor predicts 28-day mortality in patients with systemic inflammatory response syndrome. Blood 2014;123:2153-2156; Sins J W R, et al. Dynamics of von Willebrand factor reactivity in sickle cell disease during vaso-occlusive crisis and steady state. J Thromb Haemost 2017;15:1392-1402). An alternative hypothesis is that an increased WBC count leads to the formation of more platelet-leukocyte aggregates (PLAs). PLAs are associated with the release of thrombotic agents which, as hypothesized above, lead to shorter occlusion times and increase the minimum effective NAC dose (Pluta K, et al. Platelet-Leucocyte Aggregates as Novel Biomarkers in Cardiovascular Diseases. Biology (Basel). 2022;11.

### Clinical Implications

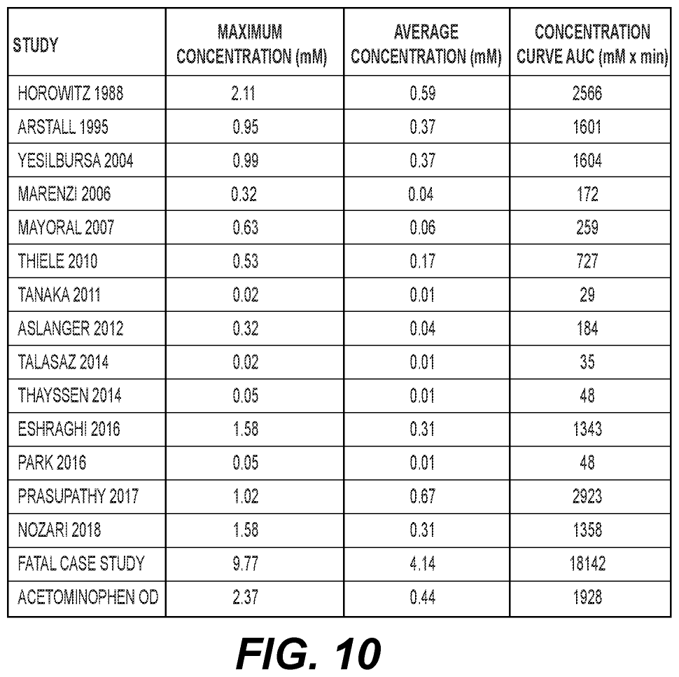

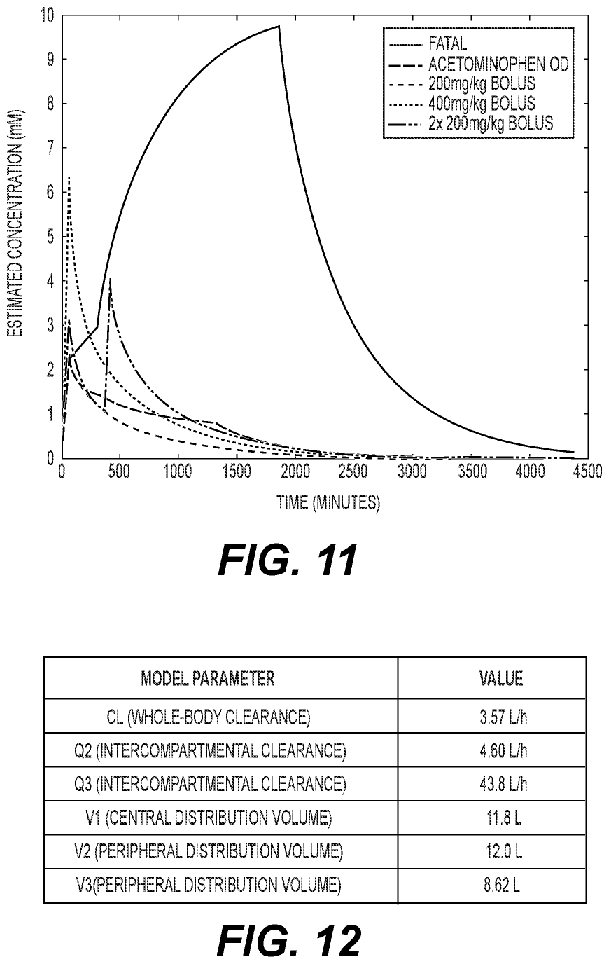

NAC is a promising therapeutic for preventing arterial thrombosis because it is a widely available, inexpensive drug with a known safety profile and established pharmacokinetics. These results from the mice tested 6 hrs after a 400 mg/kg bolus show the protective effects persist, even after the concentration of NAC in blood drops below 10 mM. Assuming that prevention is due to the permanent modification of VWF, it would be expected that the effect of NAC to decrease as altered VWF is cleared and replaced. Additionally, it was demonstrated that the effect of multiple low doses of NAC is cumulative, meaning that reaching a plasma concentration of 10 mM might not be necessary if NAC is delivered as an intravenous infusion. A simple 3-compartment model suggests that the intravenous dosing schedule currently recommended for acetaminophen overdose reaches maximal concentrations of 2.37 mM (FIG. 10). While NAC is available as oral supplements, it is impractical to reach a plasma concentration of 10 mM through ingestion because of the low oral bioavailability of NAC (Olsson B, et al. Pharmacokinetics and bioavailability of reduced and oxidized N-acetylcysteine. Eur J Clin Pharmacol 1988;34:77-82). An oral bioavailability of 4-9% means that 90 g of NAC (90× 1000 mg pills or 100× 900 mg effervescent tablets) would need to be ingested to reach plasma concentrations of 10 mM. Given the reported nausea, vomiting, diarrhea, flatus and gastro-esophagel reflux associated with lower doses of oral NAC, these high oral doses are likely infeasible. However, since it has been shown herein that the effect of NAC is lasting and cumulative, after primary treatment with an I.V. infusion a lower maintenance dose might be achieved orally. There have been reported occasions of anaphylactoid reactions to intravenous NAC and patients would need to be monitored to address these cases if they occur (Chen J, et al. N-Acetylcysteine Treatment in Two Patients with Relapsed Thrombotic Thrombocytopenia Purpura Increased ADAMTS13 Activity, Free Thiol Concentration in Plasma, and Inhibited Platelet Activation. Blood 2015;126:239-239; Heard K, et al. Massive acetylcysteine overdose associated with cerebral edema and seizures. Clin Toxicol. 2011;49:423-425). A first clinical use of NAC may involve intravenous infusion in a hospital setting, such as preventing secondary MACE after a primary event for the duration of the hospital stay.

### Potential Mechanisms

Our recovery study points to NAC preventing arterial thrombosis through modifying VWF. Previous work has demonstrated the critical role platelet VWF plays in arterial thrombosis formation. The high local concentration of platelet vWF suggests that to cause a significant effect on arterial thrombosis, the NAC needs to be in excess prior to platelet activation, and available to react with platelet VWF as it is released (Kim D A, et al. Platelet α-granules are required for occlusive high-shear-rate thrombosis. Blood Adv. 2020;4:3258-3267). NAC acting on platelet VWF would also explain our observation that while NAC has a small effect on lag time, it primarily increases occlusion time by lowering rapid platelet accumulation rates. In addition to causing longer occlusion times, a low dose of NAC in vivo led to the formation of unstable clots, suggesting that NAC also influences the mechanical properties of thrombi.

Further work is required to characterize the mechanism through which NAC can completely prevent arterial thrombosis. vWF is an unusually cysteine-rich protein that can interact with NAC in complex ways. However, there are three mechanisms of prevention suggested by the current literature. One is that NAC reduces the disulfide bonds polymerizing vWF, shortening the average multimer length (Chen J, et al. N-acetylcysteine reduces the size and activity of von Willebrand factor in human plasma and mice. J Clin Invest. 2011;121:593-603). Another is that NAC can reduce the disulfide bond linking the ends of the A1 domain critical to vWF: platelet binding. A third mechanism is that vWF-vWF self-association could be decreased by NAC disrupting the disulfide bond in the A2 domain (Solecka B A, et al. Free thiol groups in von Willebrand factor (VWF) are required for its full function under physiological flow conditions. Thromb Res. 2016;137:202-210). Also complicating the determination of the mechanism of NAC-mediated prevention of arterial thrombosis is the fact that NAC can be quickly transformed into glutathione in the blood. As another free thiol, glutathione could perform any of functions mentioned above. In reality, several modifications are likely happening simultaneously, and the current study is unable to distinguish the relative contributions of each modification. However, previous work measuring the degree of MW reduction in vWF following treatment with similar NAC concentrations and recent computational modeling of aggregate capture under varying vWF conditions suggests that length modifications alone are insufficient to cause the complete loss of platelet capture (Liu Z L, et al. SIPA in 10 milliseconds: VWF tentacles agglomerate and capture platelets under high shear. Blood Adv. 2022;6:2453-2465). While the western blots show a significant reduction in HMW multimers, the computational model suggests that vWF needs to be cleaved to a length of ˜6 dimers to cause complete prevention of platelet aggregation. This supports the hypothesis that altering the secondary structure of the

A domains is critical to attain the results seen with 10 mM NAC. Additional experiments are necessary to distinguish these mechanisms and identify the critical pathway.

### Summary

In conclusion, it is demonstrated that NAC is effective at decreasing platelet accumulation rates in a dose-dependent manner without significantly increasing bleeding in mice. NAC can completely prevent occlusive thrombi from forming at plasma concentrations of 10 mM, in human whole blood in vitro. This trend is also observed in mice using the modified-Folts model where we demonstrated prevention of occlusive thrombi and a decrease in clot stability with lower doses of NAC. NAC treatment can cause reduced clot stability for up to 6 hrs. in mice and complete prevention can be achieved through multiple injections of low concentration doses. These results suggest that NAC can be re-purposed into a potent anti-thrombotic medication.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method of preventing arterial thrombosis in a subject in need thereof, comprising: administering to the subject a thiol containing compound.

2. The method of claim 1, wherein the thiol containing compound is N-acetyl cysteine, cysteine, dithiothreitol, glutathione, dimercaptosuccinic acid, thioterpinol, methanethiol, ethanethiol, a peptide comprising N-acetyl cysteine, or any combination thereof.

3. (canceled)

4. (canceled)

5. The method of claim 1, wherein the thiol containing compound is administered at from 1 to 10 mM.

6. (canceled)

7. The method of claim 1, wherein the thiol containing compound is administered at from 100 to 1000 mg/kg dose.

8. (canceled)

9. The method of claim 1, wherein the thiol containing compound is administered once.

10. The method of claim 1, wherein the subject is at high risk of forming arterial thrombi by having a blood concentration of vWF in an upper 4^{th} quartile.

11. The method of claim 1, wherein the subject is hospitalized for cardiovascular disease.

12. The method of claim 1, wherein the subject is undergoing angioplasty or percutaneous coronary intervention, has a heart attack, or has ischemic stroke.

13. A method of treating arterial thrombosis in a subject in need thereof, comprising:
administering to the subject a thiol containing compound.

14. The method of claim 13, wherein the thiol containing compound is N-acetyl cysteine, cysteine, dithiothreitol, glutathione, dimercaptosuccinic acid, thioterpinol, methanethiol, ethanethiol, a peptide comprising N-acetyl cysteine, or any combination thereof.

15. (canceled)

16. (canceled)

17. The method of claim 13, wherein the thiol containing compound is administered at from 1 to 10 mM.

18. (canceled)

19. The method of claim 13, wherein the thiol containing compound is administered at from 100 to 1000 mg/kg dose.

20. (canceled)

21. The method of claim 13, wherein the thiol containing compound is administered once.

22. The method of claim 13, wherein the subject is at high risk of forming arterial thrombi by having a blood concentration of vWF in an upper 4^{th} quartile.

23. The method of claim 13, wherein the subject is hospitalized for cardiovascular disease.

24. The method of claim 13, wherein the subject is undergoing angioplasty or percutaneous coronary intervention, has a heart attack, or has ischemic stroke.

25. A method of reducing thrombi formation in an artery, comprising: administering to the artery an effective amount of a thiol containing compound.

26. The method of claim 25, wherein the thiol containing compound is N-acetyl cysteine, cysteine, dithiothreitol, glutathione, dimercaptosuccinic acid, thioterpinol, methanethiol, ethanethiol, a peptide comprising N-acetyl cysteine, or any combination thereof.

27. (canceled)

28. (canceled)

29. The method of claim 25, wherein the thiol containing compound is administered at from 1 to 10 mM.

30. (canceled)

31. The method of claim 25, wherein the thiol containing compound is administered at from 100 to 1000 mg/kg dose.

32. (canceled)

33. The method of claim 25, wherein the thiol containing compound is administered once.

34. The method of claim 25, wherein the artery is in a subject at high risk of forming arterial thrombi by having a blood concentration of vWF in an upper 4^{th} quartile.

35. The method of claim 25, wherein the artery is in a subject hospitalized for cardiovascular disease.

36. The method of claim 25, wherein the artery is in a subject undergoing angioplasty or percutaneous coronary intervention, has a heart attack, or has ischemic stroke.

37-39. (canceled)
